(19) United States
(12) Patent Application Publication
(10) Pub. No.: US 2004/0153125 A1
(43) Pub. Date: Aug. 5, 2004

(54) Antimicrobial fatty acid containing suture coating

(76) Inventor: Mark Roby, Killingworth, CT (US)

Correspondence Address: Patent Counsel, United States Surgical, a division of, TYCO HEALTHCARE GROUP LP, 150 Glover Avenue, Norwalk, CT, 06856, US

(21) Appl. No.: 10/728,217

(22) Filed: Dec. 4, 2003

Related U.S. Application Data
(60) Provisional application No. 60/433,528, filed on Dec. 13, 2002.

(51) Int. Cl. A61B 17/04

(52) U.S. Cl. 606/228

(57) ABSTRACT

Compositions with antimicrobial properties contain a metal salt of a fatty acid mixed with a bioabsorbable copolymer. These compositions are useful in forming coatings for surgical articles, including multifilament sutures.

### BACKGROUND

1. Technical Field

Coated surgical sutures having improved antimicrobial properties and a method for using these sutures are described. More particularly, surgical sutures including multifilament sutures coated totally or in part with mixtures of caprolactone containing copolymers and silver stearate or other non-silver stearates are provided.

2. Background of Related Art

Synthetic absorbable multifilament sutures are well known in the industry. Examples of these sutures include Dexon®, Vicryl®, and Polysorb®, commercially available from Ethicon, Inc. (Somerville, N.J.), and United States Surgical (Norwalk, Conn.).

It is known that suture materials are often coated with various substances to improve their handling characteristics. For example, U.S. Pat. Nos. 5,123,912, 4,080,969, 4,043,344, 4,047,533, and 4,027,676 describe coated surgical sutures with improved knot tie down properties.

The use of fatty acid salts in medical coatings is known. For example, U.S. Pat. No. 5,817,129 describes a process for coating sutures with a mixture of biocompatible polymer and a fatty acid salt having from 12 to 22 carbon atoms. The process can be used on monofilament sutures as well as on multifilament sutures. U.S. Pat. No. 5,304,205 describes a surgical filament that is surface coated with a mixture of N-long chain monoacylated basic amino acids and metal salts of fatty acids having at least 6 carbon atoms. U.S. Pat. No. 5,104,398 describes a multifilament suturing thread coated with a solution of a metal salt of a fatty acid having six or more carbon atoms. U.S. Pat. No. 5,019,096 describes a coating for medical devices including sutures, the coating being a mixture of dissolved matrix-forming polymer and an antimicrobial silver salt. The silver salt may be the silver salt of a fatty acid such as silver laurate or silver palmitate. U.S. Pat. No. 4,185,637 describes a multifilament suture coated with a gelled polyvalent metal ion salt of a fatty acid having 6 or more carbon atoms. U.S. Pat. No. 5,716,376 discloses an epsilon-caprolactone copolymer mixture blended with fatty acid ester to provide an absorbable suture coating mixture having improved performance characteristics.

In the early 1970's Ethicon introduced uncoated Vicryl®; see for example Horton C. E., Adamson J. E., Mladick R. A., et al: “Vicryl Synthetic Absorbable Sutures”; Am Surg, December 1974, pp 72930-31. However, this uncoated braided multifilament caused tissue trauma (tissue drag) and handling problems. As a result, in the late 1970's a Vicryl® suture coated with a glycolide/lactide copolymer blended with calcium stearate was introduced; see for example Saunder's R. A. et al: “Coated Vicryl Suture in Extraocular Muscle Surgery”. Ophthalmic Surg 10:13-8, Jul. 1979 and Kobayashi H et al. “Coated Polyglactin 910—a New Synthetic Absorbable Suture”. Jpn J Surg 11 (6):467-75, November 1981. U.S. Pat. No. 4,201,216 describes a glycolide/lactide copolymer blended with calcium stearate as a suture coating.

Although calcium stearate was used as a component in the Vicryl® suture coating, the manufacture and application of such a suture coating utilizes an impractical and uneconomical dip coating process because calcium stearate (a hydrophobic metal salt of a fatty acid) generally is water insoluble. Therefore, a suture coating fabricated from materials that would dissolve in solution and thus obviate the necessity of using dip coating processes would provide manufacturing advantages.

An important feature of a suture coating is its ability to enhance the suture's handling characteristics, such as surgeon's throw, lubricity, knot run down and/or knot security. Although commercially available surgical sutures such as Polysorb have excellent handling characteristics; it would be advantageous to provide a coated suture exhibiting even better surgeon's throw, lubricity, knot run down, and/or knot security properties.

Yet another important feature of certain suture coatings is the ability to impart antimicrobial properties to the coated suture thus providing prolonged protection against infection at the implant site. It is known to coat surgical articles, including sutures, with metallic compounds to impart antimicrobial characteristics to the articles. The anti-microbial effects of metallic ions including Ag, Au, Pt, Pd, Ir, Cu, Sn, Sb, Bi and Zn are known (see Morton, H. E., Pseudomonas in Disinfection, Sterilization and Preservation, ed. S. S. Block, Lea and Febiger, 1977 and Grier, N., Silver and Its Compounds in Disinfection, Sterilization and Preservation, ed. S. S. Block, Lea and Febiger, 1977). Silver is one of the preferred metallic ions, due to its unusually good bioactivity at low concentrations. In modern medical practice both inorganic and organic soluble salts of silver are used to prevent and treat microbial infections. While these compounds are effective as soluble salts, they do not provide prolonged protection and must be frequently reapplied. Reapplication may not always be practical, especially where an implanted device is involved. U.S. Pat. No. 6,017,553 attempts to improve upon the use of silver as an antimicrobial agent for medical devices by creating atomic disorder during vapor deposition of the metallic antimicrobial agents.

Sutures having the combined desirable properties of improved handling characteristics and antimicrobial activity, that are inexpensive and can be constructed with biocompatible materials without being subject to excessive diffusion, are desirable. This is especially so where the suture is absorbable and there is no opportunity to reapply the antimicrobial coating.

### SUMMARY

An antimicrobial coating for surgical articles is formed from a copolymer having a predominant amount of epsilon-caprolactone and a minor amount of at least one other copolymerizable monomer, and an effective antimicrobial amount of a fatty acid salt of lithium, rubidium, cesium, francium, copper, silver, gold, beryllium, magnesium, strontium, barium, radium, aluminum, tin, lead, bismuth, transition metal and mixtures thereof

In a further embodiment, a surgical suture is provided having one or more filaments of bioabsorbable material coated with a composition that is a mixture of a copolymer that is the reaction product obtained by polymerizing a predominant amount of epsilon-caprolactone and a minor amount of at least one other bioabsorbable copolymerizable monomer. Examples of other copolymerizable monomers include glycolide, trimethylene carbonate, tetramethylene carbonate, dimethyl trimethylene carbonate; dioxanones; dioxepanones; absorbable cyclic amides; absorbable cyclic ether-esters derived from crown ethers; hydroxyacids capable of esterification, including both alpha hydroxyacids (such as glycolic acid and lactic acid) and beta hydroxyacids (such as beta hydroxybutyric acid and gamma hydroxyvaleric acid); polyalkyl ethers (such as polyethylene glycol and polylpropyline glycol and combinations thereof) in the presence of polyhydric alcohol as initiator; and an effective antimicrobial amount of a fatty acid salt of lithium, rubidium, cesium, francium, beryllium, magnesium, strontium, barium, radium, aluminum, tin, lead, bismuth, transition metal and mixtures thereof.

In yet a further embodiment, a method of suturing a wound is provided. The method includes the steps of providing a sterilized needled suture, the suture being coated with the above-described antimicrobial coating, and passing the needled suture through tissue to create wound closure.

In yet a further embodiment, an implantable medical device is provided which has a coating formed from a copolymer having a predominant amount of epsilon-caprolactone and a minor amount of at least one other copolymerizable bioabsorbable monomer, and an effective antimicrobial amount of a fatty acid salt of lithium, rubidium, cesium, francium, beryllium, magnesium, strontium, barium, radium, aluminum, tin, lead, bismuth, transition metal and mixtures thereof

### BRIEF DESCRIPTION OF THE DRAWING

Various embodiments are described herein with reference to the drawing, wherein

FIG. 1 is a perspective view of a coated suture attached to a needle described herein.

### DETAILED DESCRIPTION OF PREFERRED EMBODIMENTS

It has been found that fatty acid metal salts and bioabsorbable polymers, especially those containing caprolactone, can advantageously be mixed to form a composition useful in coating implantable surgical articles, e.g., surgical sutures, medical devices, etc. to impart antimicrobial characteristics to the surgical article. It should be understood that implantable surgical articles can be formed of absorbable materials, nonabsorbable materials, and combinations thereof. Therefore, any implanable surgical article is envisioned as being suitable for use with the coating provided herein. Such a coating provides the combined desirable properties of improved handling characteristics and antimicrobial activity. Sutures coated as described herein provide the combined desirable properties of improved handling characteristics and antimicrobial activity.

Preferably, mixtures useful in forming the aforementioned coatings include a fatty acid metal salt as a predominant component in an effective antimicrobial amount. A “predominant amount” refers to one or more components which are present in an amount greater than about 50 weight percent. A “minor amount” refers to one or more components which are present in an amount up to about 50 weight percent. The minor component includes copolymers containing caprolactone.

An “effective antimicrobial amount” of a given component is an amount at which the component hinders the growth of bacteria to diminish or avoid contamination of the wound site.

Preferably, the antimicrobial absorbable coating composition for biocompatible surgical implantable devices is inexpensive, biocompatible, and not subject to excessive diffusion. “Biocompatible” means that no serious systemic toxicity is caused by the presence of an object in a living system. It is contemplated that biocompatible objects may cause some clinically acceptable amounts of toxicity including irritation and/or other adverse reactions in certain individuals. In a particularly useful embodiment, the antimicrobial absorbable coating composition is applied to multifilament synthetic surgical sutures.

An example of suitable fatty acid metal salts useful as the antimicrobial agent in the various embodiments herein are metal stearates. In one embodiment, the fatty acid metal salt used as the antimicrobial agent is silver stearate. In another embodiment, the fatty acid metal salt(s) used as the antimicrobial agent may be combined with fatty acid esters such as stearoyl lactylates, particularly calcium stearoyl lactylate.

Any bioabsorbable polymer known to those skilled in the art can be employed in the present coatings. In particularly useful embodiments, the bioabsorbable polymer contains epsilon-caprolactone as a component thereof Suitable caprolactone containing copolymers include copolymers which may be synthesized by well known conventional polymerization techniques; see, for example Principles of polymerization, George Odian, III Edition; 1991 pp. 569-573, the contents of which are incorporated herein by reference. Particularly useful caprolactone containing copolymers are “star” copolymers obtained by polymerizing a predominant amount of epsilon-caprolactone and a minor amount of another bioabsorbable monomer polymerizable therewith in the presence of a polyhydric alcohol initiator.

Preferably, the caprolactone containing copolymer is obtained by polymerizing a predominant amount of epsilon-caprolactone and a minor amount of at least one other copolymerizable monomer or mixture of such monomers in the presence of a polyhydric alcohol initiator. The polymerization of these monomers contemplates all of the various types of monomer addition, i.e., simultaneous, sequential, simultaneous followed by sequential, sequential followed by simultaneous, etc.

In certain embodiments, the copolymer herein can contain from about 70 to about 98, and preferably from about 80 to about 95, weight percent epsilon-caprolactone derived units, the balance of the copolymer being derived from the other copolymerizable monomer(s).

Suitable monomers which can be copolymerized with epsilon-caprolactone include alkylene carbonates such as trimethylene carbonate, tetramethylene carbonate, dimethyl trimethylene carbonate; dioxanones; dioxepanones; absorbable cyclic amides; absorbable cyclic ether-esters derived from crown ethers; hydroxyacids capable of esterification, including both alpha hydroxyacids (such as glycolic acid and lactic acid) and beta hydroxyacids (such as beta hydroxybutyric acid and gamma hydroxyvaleric acid); polyalkyl ethers (such as polyethylene glycol and polyloropyline glycol and combinations thereof); with glycolide being a preferred monomer.

Suitable polyhydric alcohol initiators include glycerol, trimethylolpropane, 1,2,4-butanetriol, 1,2,6-hexanetriol, triethanolamine, triisopropanolamine, erythritol, threitol, pentaerythritol, ribitol, arabinitol, xylitol, N,N,N′,N′-tetrakis(2-hydroxyethyl)ethylenediamine, N,N,N′,N′-tetrakis(2-hydroxypropyl)ethylenediamine, dipentaerythritol, allitol, dulcitol, glucitol, altritol, iditol, sorbitol, mannitol, inositol, and the like; with mannitol being preferred.

The polyhydric alcohol initiator is generally employed in relatively small amounts, e.g., from about 0.01 to about 5, and preferably from about 0.1 to about 3, weight percent of the total monomer mixture.

The coating composition can contain from about 0.3 to about 10, and preferably from about 0.5 to about 5, weight percent of the copolymer.

Suitable fatty acids which can be used in the present coatings include the biocompatible monovalent and polyvalent metal salts of fatty acids having 6 or more carbon atoms. Examples of fatty acids useful for forming a metal salt of a fatty acid useful herein includes butyric, caproic, caprylic, capric, lauric, myristic, palmitic, palmitoleic, stearic, oleic, linoleic, linolenic, etc. Examples of monovalent metals useful for forming a metal salt of a fatty acid useful in the various embodiments described herein include lithium, rubidium, cesium, francium, copper, silver and gold. Examples of polyvalent metals useful for forming a metal salt of a fatty acid useful in the various embodiments described herein include aluminum, tin, lead, bismuth and the polyvalent transition metals. Therefore, suitable metal salts of fatty acids useful herein include fatty acid salts of lithium, rubidium, cesium, francium, copper, silver, gold, beryllium, magnesium, strontium, barium, radium, aluminum, tin, lead, bismuth, zinc, cadmium, mercury, etc.

The metal salt of a fatty acid is present in the coating composition in an effective antimicrobial amount as defined above. The metal salt of a fatty acid can consist of a single chemical compound. However, the metal salt of a fatty acid can also be a mixture of several metal salts of fatty acids. Typically, the metal salt of a fatty acid is present in an amount from about 30 percent to about 70 percent by weight of the coating composition. Preferably, the metal salt of a fatty acid is present in an amount from about 45 percent to about 55 percent by weight of the coating composition.

The metal salt of a fatty acid may be relatively insoluble in cold water. When desirable, a solvent may be used to improve the working properties, e.g., viscosity, miscability, etc., of the metal salt of a fatty acid. Suitable solvents include, for example, alcohols, e.g., methanol, ethanol, propanol, chlorinated hydrocarbons (such as methylene chloride, chloroform, 1,2-dichloro-ethane), aliphatic hydrocarbons such as hexane, heptene, ethyl acetate). When desirable, heat may be applied to the solvent mixture of metal salts of fatty acids to improve their solubility. For example, temperatures ranging from about 30° C. to about 60° C. are appropriate.

The caprolactone containing copolymer and the metal salt of a fatty acid are biocompatible; a mixture of the two is biocompatible as well. In certain embodiments, fatty acid esters are combined with the metal salt of a fatty acid in the coating composition. Such esters include, for example, calcium stearate, stearoyl lactylate esters, palmityl lactylate esters, oleyl lactylate esters such as calcium, magnesium, aluminum, barium, or zinc stearoyl lactylate; calcium, magnesium, aluminum, barium, or zinc palmityl lactylate; calcium, magnesium, aluminum, barium, or zinc oleyl lactylate; with calcium stearate and calcium stearoyl-2-lactylate (such as the calcium stearoyl-2-lactylate commercially available under the tradename VERV from American Ingredients Co., Kansas City, Mo.) being preferred. When desirable, the fatty acid ester may be combined with a solvent. Suitable solvents include, those listed above.

The bioabsorbable mixture herein can be prepared by mixing the components and solvents separately and then combining the solvent mixtures to form the coating solution or by mixing the components together and then mixing with solvent to form the coating solution or any combination thereof The order of addition is not critical and therefore may be determined through routine experimentation depending upon the desired use.

The bioabsorbable mixture herein can be applied to a suture by any suitable process, e.g., passing the suture through a solution of the coating mixture, past a brush or other coating solution applicator, or past one or more spray nozzles dispensing the suture coating solution. The coating solution can contain from about 30 to about 70, preferably from about 45 to about 55, weight percent solvent. In a preferred embodiment, a mixture of methylene chloride, hexane and ethanol is used as a solvent. The suture wetted with the coating solution is optionally passed through or held in a drying oven for a time and at a temperature sufficient to vaporize and drive off the solvent. If desired, the suture coating composition can optionally contain additional components, e.g., dyes, antibiotics, antiseptics, growth factors, anti-inflammatory agents, etc.

While the coating composition herein can be applied to any type of suture, it is particularly well-suited for application to a braided suture, a preferred type of which is disclosed in U.S. Pat. No. 5,019,093. The amount of coating composition applied to a braided suture will vary depending upon the structure of the suture, e.g., the number of filaments, tightness of braid or twist, the size of the suture and its composition. Suitable coating levels can range from about 0.3% to about 10% with about 0.5% to about 5% of the weight of the suture being preferred.

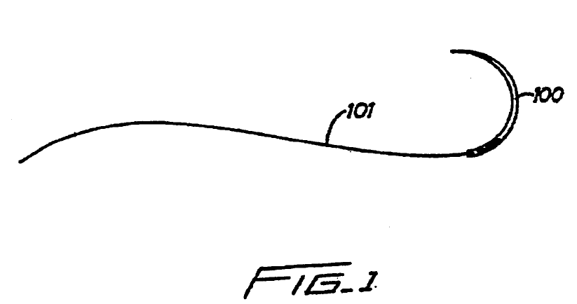

The coated suture 101 may be attached to a surgical needle 100 as shown in FIG. 1 by methods well known in the art. Wounds may be sutured by passing the needled suture through tissue to create wound closure. The needle preferably is then removed from the suture and the suture tied. The coating, in addition to enhancing the suture's handling characteristics, advantageously possesses antimicrobial properties to promote healing and prevent infection.

The following examples are given as an illustration of the preparation of certain copolymers, blends, and coatings described herein as well as the superior characteristics of certain sutures described herein. It should be noted that the various embodiments described herein are not limited to the specific details embodied in the examples.

### EXAMPLE 1

Dry glycolide (222 g), epsilon-caprolactone (2000 g), stannous octoate as catalyst (0.44 g) and dry mannitol as initiator (2.2 g) are mixed under N₂ for one hour. The mixture is heated in a reactor at a temperature of 160 [symbol] C. for 12 hours. The reaction product, an epsilon-caprolactone/glycolide star copolymer is then sampled.

1820 g of a mixture of methylene chloride, hexane and ethanol were mixed with 180 grams of the reaction product at 90 [symbol] C. for 2 hours under constant stirring, to form a solution.

### EXAMPLE 2

1820 g of a mixture of methylene chloride, hexane and ethenol is mixed with 180 grams of silver stearate at room tempeture for 2 hours under constant stirring to form a suspension.

### EXAMPLE 3

1820 g of a mixture of methylene chloride hexane and ethenol is mixed with 180 grams of calcium stearoyl lactylate (commercially available from American Ingredients Co., Kansas City, Mo., under the tradename VERV) at 20° C. for 3 hours under constant stirring to form a suspension.

### EXAMPLE 4

1000 g of the solution of Example 1 is mixed with 1000 grams of the solution of Example 2 at 20° C. for 30 minutes under constant stirring to form a coating solution.

### EXAMPLE 5

1000 g of the solution of Example 1 is mixed with 1000 grams of the solution of Example 2 and 500 grams of the solution of Example 3 at 25 [symbol] C. for 10 minutes under constant stirring to form a coating solution.

### EXAMPLE 6

A size 0 Polysorb surgical suture is drawn through a coating solution applicator to apply the coating solution of Example 4, at a level of about 2 percent by weight of the suture, to coat the suture with the coating solution.

### EXAMPLE 7

A size 0 Polysorb surgical suture is drawn through a coating solution applicator to apply the coating solution of Example 5, at a level of about 2 percent by weight of the suture, to coat the suture with the coating solution.

It will be understood that various modifications may be made to the embodiments disclosed herein. For example, although it is preferred to coat surgical sutures from the disclosed coating mixtures, a wide variety of surgical articles can be coated. These include but are not limited to surgical clips and other fasteners, staples, pins, screws, prosthetic devices, drug delivery devices, meshes or fabrics, anastomosis rings, and other implantable devices. Therefore, the above description should not be construed as limiting, but merely as exemplifications of preferred embodiments. Those skilled in the art will envision other modifications within the scope and spirit of the claims appended hereto.

What is claimed is:

1. A coating for a surgical article comprising:
a) a copolymer having a predominant amount of epsilon-caprolactone and a minor amount of at least one other bioabsorbable copolymerizable monomer; and
b) an effective antimicrobial amount of a metal salt of a fatty acid selected from the group consisting of fatty acid salts of lithium, rubidium, cesium, francium, beryllium, magnesium, strontium, barium, radium, aluminum, tin, lead, bismuth, transition metal and mixtures thereof.

2. The coating for a surgical article of claim 1 wherein the metal salt of a fatty acid is silver stearate.

3. The coating for a surgical article of claim 1 further including a fatty acid ester.

4. The coating for a surgical article coating of claim 3 wherein the fatty acid ester is selected from the group consisting of calcium stearoyl lactylate, magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, zinc oleyl lactylate and mixtures thereof.

5. The coating for a surgical article of claim 3 wherein the fatty acid ester is calcium stearoyl lactylate.

6. The coating for a surgical article of claim 1 wherein the metal salt of a fatty acid ranges from about 0.3 to about 10 percent by weight of the coating composition.

7. The coating for a surgical article of claim 3 wherein the fatty acid ester ranges from about 30 to about 70 percent by weight of the coating composition.

8. The coating for a surgical article of claim 1 wherein the surgical article is a suture.

9. A surgical suture having one or more filaments of bioabsorbable material coated with a composition comprising a copolymer having a predominant amount of epsilon-caprolactone and a minor amount of at least one other bioabsorbable copolymerizable monomer and an effective antimicrobial amount of a metal salt of a fatty acid selected from the group consisting of fatty acid salts of lithium, rubidium, cesium, francium, beryllium, magnesium, strontium, barium, radium, aluminum, tin, lead, bismuth, transition metal and mixtures thereof.

10. The surgical suture of claim 9 wherein the suture is a braided suture.

11. The surgical suture of claim 9 wherein the metal salt of a fatty acid is silver stearate.

12. The surgical suture of claim 9 wherein the composition further includes a fatty acid ester.

13. The surgical suture of claim 12 wherein the fatty acid ester is selected from the group consisting of calcium stearoyl lactylate, magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, zinc oleyl lactylate and mixtures thereof.

14. The surgical suture of claim 12 wherein the fatty acid ester is calcium stearoyl lactylate.

15. The surgical suture of claim 11 wherein calcium stearoyl lactylate is combined with the silver stearate.

16. The surgical suture of claim 9 wherein the copolymer contains from about 80 to about 95 percent by weight epsilon-caprolactone.

17. A method of suturing a wound comprising:
a) providing a sterilized needled suture, said suture coated with a composition that is a mixture of:
1) a copolymer that is the reaction product obtained by polymerizing a predominant amount of epsilon-caprolactone and a minor amount of at least one other bioabsorbable copolymerizable monomer-selected from the group consisting of alkylene carbonates, dioxanones, dioxepanones, absorbable cyclic amides, absorbable cyclic ether-esters derived from crown ethers, hydroxyacids capable of esterification and mixtures thereof; and
2) an effective antimicrobial amount of a metal salt of a fatty acid selected from the group consisting of fatty acid salts of lithium, rubidium, cesium, francium, beryllium, magnesium, strontium, barium, radium, aluminum, tin, lead, bismuth, transition metal and mixtures thereof; and
b) passing the needled suture through tissue to create wound closure.

18. The method of claim 17 wherein the metal salt of a fatty acid is silver stearate.

19. The method of claim 17 wherein the composition further contains a fatty acid ester.

20. The method of claim 18 wherein the fatty acid ester is selected from the group consisting of calcium stearoyl lactylate, magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, zinc oleyl lactylate and mixtures thereof.

21. The method of claim 17 wherein calcium stearoyl lactylate is combined with the metal salt of a fatty acid.

22. An implantable medical device having a coating comprising a mixture of:
a) a copolymer containing a predominant amount of epsilon-caprolactone and a minor amount of glycolide; and
b) an effective antimicrobial amount of a metal salt of a fatty acid, the metal salt of a fatty acid selected from the group consisting of fatty acid salts of lithium, rubidium, cesium, francium, beryllium, magnesium, strontium, barium, radium, aluminum, tin, lead, bismuth, transition metal and mixtures thereof.

23. The implantable medical device of claim 22 wherein the coating further comprises a fatty acid ester.

24. The implantable medical device of claim 23 wherein the fatty acid ester is selected from the group consisting of calcium stearoyl lactylate, magnesium stearoyl lactylate, aluminum stearoyl lactylate, barium stearoyl lactylate, zinc stearoyl lactylate, calcium palmityl lactylate, magnesium palmityl lactylate, aluminum palmityl lactylate, barium palmityl lactylate, zinc palmityl lactylate, calcium oleyl lactylate, magnesium oleyl lactylate, aluminum oleyl lactylate, barium oleyl lactylate, zinc oleyl lactylate and mixtures thereof.

25. The implantable medical device of claim 23 wherein the coating includes calcium stearoyl lactylate and silver stearate.

26. The implantable medical device of claim 22 wherein the medical device is selected from the group consisting of clips, staples, pins, screws, prosthetic devices, anastomosis rings, and growth matrices.
